(19) United States
(12) Patent Application Publication
(10) Pub. No.: US 2014/0159725 A1
(43) Pub. Date: Jun. 12, 2014

(54) METHOD AND ARRANGEMENT FOR CHARACTERIZED TISSUE OF HUMAN OR ANIMAL TISSUE

(71) Applicants: Ingolf Sack, Berlin (DE); Jürgen Braun, Berlin (DE); Sebastian Papazoglou, Berlin (DE); Sebastian Hirsch, Berlin (DE)

(72) Inventors: Ingolf Sack, Berlin (DE); Jürgen Braun, Berlin (DE); Sebastian Papazoglou, Berlin (DE); Sebastian Hirsch, Berlin (DE)

(73) Assignee: Charite-Universitatsmedizin Berlin, Berlin (DE)

(21) Appl. No.: 14/114,029

(22) PCT Filed: Apr. 20, 2012
(86) PCT No.: PCT/DE2012/200028

§ 371 Date: Dec. 4, 2013

(30) Foreign Application Priority Data
Apr. 29, 2011 (DE) 10 2011 017 778.7

(51) Int. Cl. G01R 33/54; A61B 8/08

(52) U.S. Cl. CPC G01R 33/54; A61B 8/52
USPC 324/309; 600/411; 600/437

(57) ABSTRACT

The invention relates to, inter alia, a method for characterizing tissue of human or animal tissue, wherein a vector field (u) is established, which specifies the mechanical deflection or a time derivative of the mechanical deflection of tissue particles present in the tissue, the divergence of the vector field (∇·u) is determined and the divergence of the vector field is used as a measurement result characterizing the tissue for the purposes of tissue characterization.

The invention relates to a method for characterizing tissue of human or animal tissue.

It is well known that, in addition to morphological imaging, magnetic resonance imaging (MRI) allows the display of a number of functional and constitutive variables in the living organism. In addition to the typical radiological contrast parameters, which are based on relaxation time differences between body-own tissue types and liquids, functional MRI, diffusion-weighted MRI, MR (magnetic resonance) angiography, susceptibility, perfusion and flow imaging and MR elastography are of importance. The two last-mentioned techniques are based on recording coherent three-dimensional (3D) motion fields which can be generated extrinsically by means of wave stimulation in the tissue or in the vessel system or which occur intrinsically due to the heart beating, blood flow, respiration, etc.

Flow imaging and elastography are currently the only applications of vector field MRI. Flow imaging is applied clinically to the heart and in vessels in order to quantify flow velocities. Elastography is currently evaluated clinically for graduating hepatic fibrosis, diastolic heart dysfunction and neurodegenerative processes. Quantifiable measurement units in flow imaging are velocities in m/s, while the shear modulus of the tissue is determined by means of MR elastography.

The invention is based on the object of specifying a method which can supply measurement results which go beyond the aforementioned measurement results.

According to the invention, this object is achieved by a method with the features in accordance with patent claim 1. Advantageous embodiments of the method according to the invention are specified in the dependent claims.

According to this, provision is made according to the invention for a vector field to be established, which specifies the mechanical deflection or a time derivative of the mechanical deflection of tissue particles present in the tissue, for the divergence of the vector field to be determined and the divergence of the vector field to be used as a measurement result characterizing the tissue.

A substantial advantage of the method according to the invention consists in the evaluation of the divergence of the vector field, which is provided for according to the invention. By way of example, the evaluation of the divergence of the vector field allows pressure to be established in a very advantageous fashion and, with this, in particular the identification of edemas, steatoses, vascular occlusions, hypertonia or metabolic dysfunctions.

In accordance with a particularly preferred embodiment of the method, provision is made, using the divergence of the vector field, for at least one measurement value to be established, which describes a local volume change in the examined tissue.

It is preferable, using the divergence of the vector field, to establish at least one measurement value, which has the dimension of pressure. By way of example, the measurement value can—as already mentioned—be a pressure measurement value.

It is considered to be particularly simple and hence advantageous if the phase of a measurement signal from an imaging apparatus is evaluated and the vector field is determined using the phase signal.

The measurement value preferably specifies a local volume or pressure change in the tissue which is based on an intrinsic pressure change. Alternatively, or in addition thereto, the tissue can be stimulated externally and a measurement value which specifies a local volume or pressure change in the tissue based on the external simulation can be formed.

In the case of a harmonic tissue vibration of the tissue particle with the frequency f and a sinusoidal motion encoding gradient with N cycles, duration τ and gradient amplitude g, the vector field u is preferably established using a phase signal, which for example specifies the signal phase φ of a measurement signal from an imaging apparatus, in accordance with the following relation:

$$u = ϕ \cdot \vert \frac{π ( 1 - {τ}^{2} {f}^{2} )}{γ g τsin ( π N τ f )} \vert$$

where γ denotes the gyromagnetic ratio between the magnetic moment and the spin of a proton.

It is considered to be advantageous if the measurement value is formed by multiplying the magnitude of the divergence of the vector field by a proportionality factor.

For the case that an examined tissue section can, at least approximately, be considered to be an isolated cavity, the measurement value is preferably established in accordance with the following relation:

$$p = - \frac{{p}_{0}}{{n}_{f}} ( ∇ \cdot u ) ,$$

where u denotes the vector field, n_{f} denotes a volume fraction of gas or liquid in relation to the overall volume, p₀ denotes a reference pressure and (∇·u) denotes the divergence of the vector field.

For the case that an examined tissue section, at least approximately, contains incompressible media, the measurement value is preferably established in accordance with the following relation:

$$Δ p = - {ω}^{2} ρ \frac{1 - {n}_{f}}{{n}_{f}} ( ∇ \cdot u ) ,$$

where ω denotes the angular frequency of an external mechanical stimulation and ρ denotes the density.

Alternatively, the measurement value can be formed by solving an integral equation which contains the divergence of the vector field as part of the integrand of an integral. By way of example, the measurement value is formed by solving the following integral equation:

$$p ( \vec{r} ) = - \frac{1}{4 π} {ω}^{2} ρα \int \frac{1}{\vert \vec{r} - {\vec{r}}_{0} \vert} ∇ \cdot u ( {\vec{r}}_{0} ) d V ( {\vec{r}}_{0} ) ,$$

where α describes a dimensionless scaling variable, which depends on the inherent material property of the enclosed medium.

It is considered to be particularly advantageous if the phase of a magnetic resonance imaging measurement signal is evaluated and the vector field is determined using this phase signal.

Alternatively, it is considered to be advantageous if an ultrasound signal is generated and coupled into the tissue to be examined. The vector field is preferably established by measuring and evaluating a measured back-coupled ultrasound signal.

The invention moreover relates to an arrangement for characterizing tissue of human or animal tissue. According to the invention, provision is made for the arrangement to have a computer apparatus and memory, wherein a program for controlling the computer apparatus is stored in the memory. The computer apparatus—when executing the program—is preferably suitable for determining the divergence of a vector field, which specifies the mechanical deflection or a time derivative of the mechanical deflection of tissue particles present in the tissue, and for using the divergence of the vector field as a measurement result characterizing the tissue for the purposes of tissue characterization.

The program stored in the memory is preferably suitable for actuating the computer apparatus in such a way that the computer apparatus executes a method for characterizing tissue of human or animal tissue, as described above in various variants.

The arrangement particularly preferably comprises an imaging apparatus which supplies a measurement signal, the phase of which is evaluated. The vector field is preferably determined using the phase signal.

The imaging apparatus is preferably a magnetic resonance imaging scanner, the magnetic resonance imaging measurement signal of which is evaluated. The vector field is preferably determined using the phase signal from the magnetic resonance imaging measurement signal.

Alternatively, the imaging apparatus can be an ultrasound measuring apparatus, by means of which an ultrasound signal is generated and coupled into the tissue to be examined. In this case, the vector field is preferably established by measuring and evaluating a measured back-coupled ultrasound signal.

The invention will be explained in more detail below on the basis of exemplary embodiments; here, in an exemplary fashion:

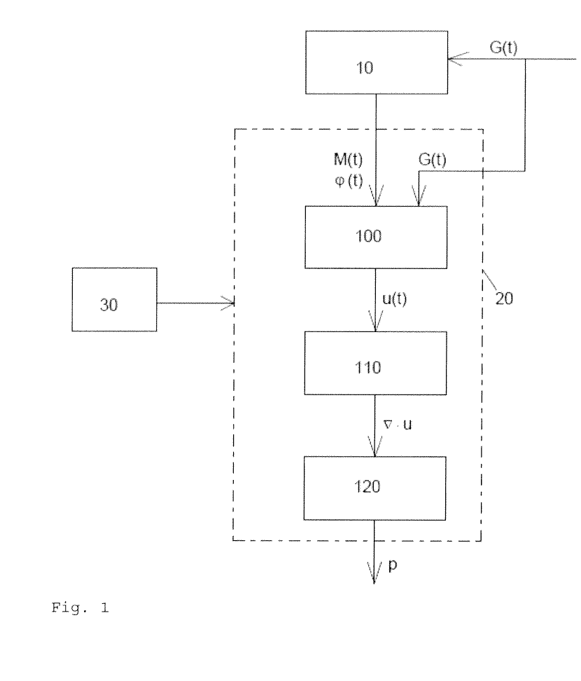

FIG. 1 shows an exemplary embodiment for an arrangement, on the basis of which an exemplary embodiment of the method according to the invention is explained,

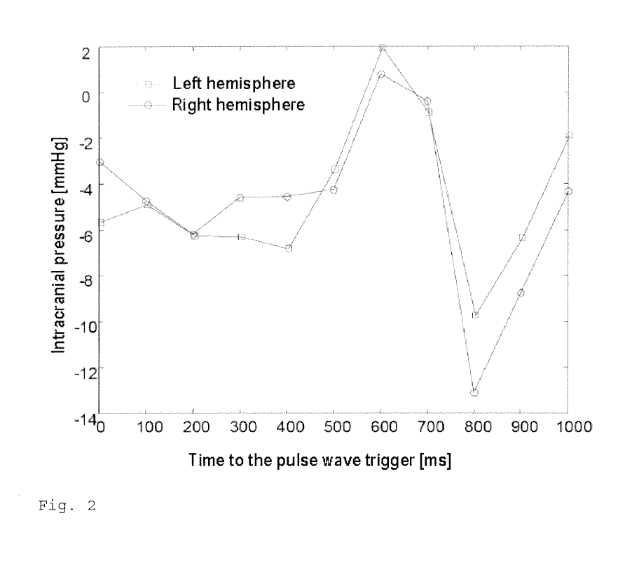

FIG. 2 plots the intracranial pressure in a healthy volunteer, established according to the method as per FIG. 1, against the cardiac pulse wave and

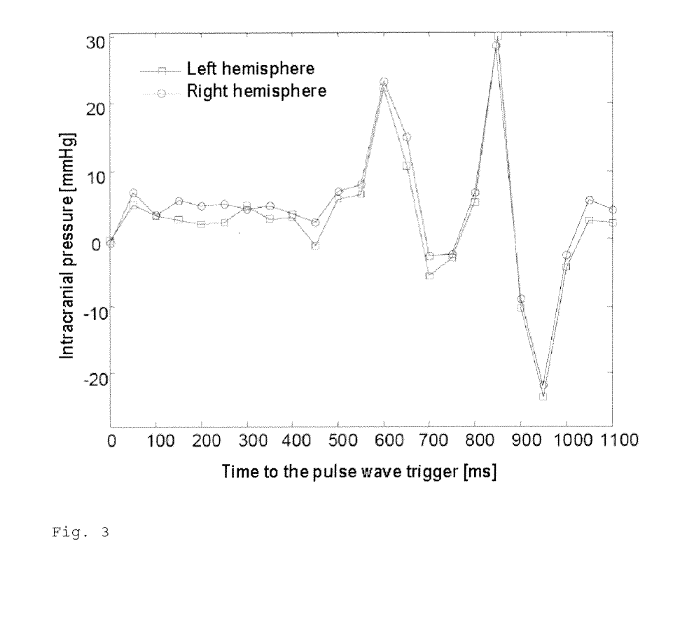

FIG. 3 plots the intracranial pressure in a healthy volunteer, established according to the method as per FIG. 1, without mechanical excitation.

In FIG. 1, it is possible to identify a medical imaging apparatus 10, which can, for example, be an MRI imaging apparatus or an ultrasound imaging apparatus. For characterizing tissue, which is not depicted in FIG. 1, the imaging apparatus 10 generates a measurement signal M(t) with a phase which is characterized by a signal phase φ(t)=(φₓ(t),φ_{y}(t),φ_{z}(t)). The measurement signal M(t) and the phase signal specifying the signal phase φ are vector quantities in this case.

A computer apparatus 20, which is connected to a memory 30, is arranged downstream from the imaging apparatus 10. Stored in the memory 30 is a program which enables the computer apparatus 20 to evaluate the signal phase φ of the measurement signal M(t) and to establish a vector field u=(uₓ(x, y, z), n_{y}(x, y, z), u_{z}(x, y, z)) which specifies the mechanical deflection or a time derivative of the mechanical deflection of one or more tissue particles present in the tissue (cf. step 100 in FIG. 1). Using the vector field u, the computer apparatus 20 determines e.g. a pressure measurement value p within the scope of further steps 110 and 120, as will be explained in more detail in an exemplary manner below.

By way of example, if the imaging apparatus 10 is an MRI scanner which carries out a phase contrast MRI method (cf. [1]), the recorded signal phase φ scales with the strength of the mechanical deflection of the tissue particles or with the time derivatives thereof. In this case, the recorded signal phase φ can for example be accumulated over the time τ of the application of a motion encoding gradient G prescribed when carrying out the MRI imaging (cf. [1]). Since G is a vector quantity, the components Gᵢ (i.e. Gₓ, G_{y} and G_{z}) of which are defined along the Cartesian axes of the MRI system, the following applies in this case:

$${ϕ}_{i} ( x , y , z , t ) = γ {\int }_{0}^{τ} {G}_{i} ( t ) {u}_{i} ( x , y , z , t ) d t \qquad ( 1 )$$

where uᵢ is any component, i.e. the x-, y- or z-component of the vector field u which specifies the mechanical deflection or a time derivative of the mechanical deflection of a tissue particle present in the tissue.

In the case of a harmonic tissue vibration of the tissue particle with the frequency f and a sinusoidal motion encoding gradient G with N cycles, duration t and gradient amplitude g, the vector field u emerges as a three-dimensional wave field as per [1] as:

$$u = ϕ \cdot \vert \frac{π ( 1 - {τ}^{2} {f}^{2} )}{γ g τsin ( π N τ f )} \vert \qquad ( 2 )$$

where γ denotes the gyromagnetic ratio between the magnetic moment and the spin of a proton.

By way of example, it is now possible to establish local volume changes by calculating the divergence of the vector field u or by calculating the divergence of a vector field formed from a time derivative of the vector field u (du/dt, d²u/dt², . . . , dⁿu/dtⁿ). Hence, it is possible to draw conclusions in respect of compressibility and pressure changes in the tissue by means of the signal phase φ. The divergence of u, (∇·u), is calculated as follows in three dimensions (cf. step 110 in FIG. 1):

$$∇ \cdot u = \frac{\partial {u}_{x}}{\partial x} + \frac{\partial {u}_{y}}{\partial y} + \frac{\partial {u}_{z}}{\partial z} \qquad ( 3 )$$

i.e. the Cartesian directional derivatives of the field are simply summated. Equation (3) provides a direct, initial expression for the local compressibility of biological tissue, which can be used directly as a diagnostic parameter. Hence it is not indispensable to convert ∇·u into physical structure or pressure variables with the aid of various model approaches. Nevertheless, the relation of the divergence in relation to tissue-inherent pressure variables should briefly be described below and the calculation thereof should be demonstrated by using equation (3).

In order to derive the tissue pressure p (unit for example Pa) from the divergence, it is necessary to solve a potential equation [2] (cf. step 120 in FIG. 1):

$$αΔ p + {n}_{f} {ω}^{2} \frac{{ρ}_{0}^{f}}{{p}_{0}} P + {ρ}_{o}^{f} {ω}^{2} ( 1 - α ) ( ∇ \cdot u ) = 0 \qquad ( 4 )$$

In equation (4), the tissue is assumed to be a biphasic medium without internal force terms. In such a way, a solid tissue matrix could enclose a compressible, possibly gaseous medium, like, for example, in the lung or in a parenchyma matrix, which is pervaded by liquid-filled vessels (brain, liver). Then u describes the vector field of the parenchyma deflection, Δ is the Laplace operator. The volume fraction of gas or liquid in relation to the overall volume is denoted by n_{f} in (4). ρ₀^{f} corresponds to the gas or liquid density at the reference pressure p₀. ω is the angular frequency of the mechanical stimulation, while α denotes a dimensionless scaling variable which depends on the inherent material property of the enclosed medium. Inter alia, α is determined by the pneumatic or hydraulic resistance of the enclosed medium, i.e. α tends to zero in the case of an infinitely high resistance, while a very low transport resistance of the enclosed medium leads to Re(α)→n_{f} and Im(α)→0. Hence, it is possible to specify two limit cases for equation (4):

$$\begin{matrix}p = - \frac{{p}_{0}}{{n}_{f}} ( ∇ \cdot u ) & ( 5 a ) \\ Δ p + {ω}^{2} \frac{{ρ}_{0}^{f}}{{p}_{0}} p = - {ω}^{2} {ρ}_{0}^{f} ( \frac{1 - {n}_{f}}{{n}_{f}} ) ( ∇ \cdot u ) & ( 5 b )\end{matrix}$$

(5a) corresponds to the case of isolated cavities and satisfies the ideal gas law, while (5b) describes the case of communicating vessels with unhindered gas or liquid exchange. The enclosed medium is compressible in both cases. Assuming incompressible materials for the matrix and the enclosed medium with density ρ, the following applies [2]

$$Δ p + {ρω}^{2} \frac{1 - α}{α} ( ∇ \cdot u ) = 0 \qquad ( 6 )$$

FIG. 2 plots, in an exemplary manner, the intracranial pressure in a healthy volunteer against the cardiac pulse wave, determined by means of divergence-based MRI in accordance with equation (6) at 25 Hz excitation frequency; a value of 1 was assumed for α.

FIG. 3 plots, in an exemplary manner, the intracranial pressure in a healthy volunteer without mechanical excitation. Since the exact motion model in the tissue is unknown without extrinsic stimulation, assumptions have to be made e.g. for ω in equation (6), which influence the absolute scaling of the pressure. Determining the absolute pressure change is only approximate for this reason but the relative intracranial pressure variations can be detected very well.

By way of example, the following values can be selected as MRI control parameters for the MRI-based measurements in accordance with FIGS. 2 and 3:
recording time of a complete 3D vector data record with 30 slices: 22 s (without time resolution) or (with time resolution) 90 s with 4 time steps or 3 minutes with 8 time steps.
voxel size 2×2×2 mm³, motion encoding gradient: 50 ms bipolar 20 mT/m, “first moment nulling” (setting the first moment to zero).

In accordance with the aforementioned limit cases, the following is obtained for incompressible media:

$$\begin{matrix}∇ \cdot u = 0 & ( 7 a ) \\ Δ p = - {ω}^{2} ρ \frac{1 - {n}_{f}}{{n}_{f}} ( ∇ \cdot u ) & ( 7 b )\end{matrix}$$

i.e. the material behaves like a monophasic incompressible medium (divergence=local volume change=zero) in the case of closed-off vessels, while ∇·u≠0 is measured in the case of communicating vessels, as is to be expected in biological tissue. Formally, equation (7b) is identical to the Poisson equation known from electrostatics, for which a closed-form (analytic) solution exists:

$$p ( \vec{r} ) = - \frac{1}{4 π} {ω}^{2} ρα \int \frac{1}{\vert \vec{r} - {\vec{r}}_{0} \vert} ∇ \cdot u ( {\vec{r}}_{0} ) d V ( {\vec{r}}_{0} ) , \qquad ( 8 )$$

Equation (8) corresponds to a simple convolution of the divergence of the motion field with 1/r.

As already explained, FIGS. 2 and 3 demonstrate the application of divergence-based MRI to healthy volunteers in order to determine intracranial pressure variations over the cardiac phase. The divergence of a motion field can, according to equation (5a) or (8), be converted into a pressure quantity with and without extrinsic stimulation.

The method of divergence-based magnetic resonance imaging, described in an exemplary manner, has the following advantages:
The method provides the option for noninvasive and image-supported determination of local pressure changes in the tissue.
The method represents a novel diagnostic modality. Local volume changes can be determined by means of the divergence operator according to equation (3).
The divergence operator generates a new image contrast, which provides an impression in relation to pressure variations in the tissue, even without further processing (e.g. according to equation (3)).

The described method was tested in compressible tissue phantoms and on the brain of healthy volunteers. The cardiac pressure wave could be quantified in the brain parenchyma, both with low-frequency mechanical stimulation (25 Hz) and under the influence of intrinsic pulsation. Pressure differences lie in the region of up to 10 mmHg, which corresponds to the physiological pressure differences in the pulsating brain. The previously compiled reference values originate from invasive methods with direct pressure measurement probes. However, for example, a noninvasive pressure determination using MRI or ultrasound is also possible on the basis of the described method.

### LITERATURE

[1] Asbach P, Klatt D, Hamhaber U, Braun J, Somasundaram R, Hamm B, Sack I. Assessment of liver viscoelasticity using multifrequency MR elastography. Magn Reson Med 2008; 60:373-379.
[2] Schanz M, Diebels S. A comparative study of Biot's theory and the linear Theory of Porous Media for wave propagation problems. Acta Mech 2003; 161(3-4):213-235.
[3] Urchuk S N, Plewes D B. MR measurement of time-dependent blood pressure variations. J Magn Reson Imaging 1995; 5(6):621-627.
[4] Miyati T, Mase M, Kasai H, Hara M, Yamada K, Shibamoto Y, Soellinger M, Baltes C, Luechinger R. Noninvasive MRI assessment of intracranial compliance in idiopathic normal pressure hydrocephalus. J Magn Reson Imaging 2007; 26(2):274-278.
[5] Song S M, Leahy R M, Boyd D P, Brundage B H, Napel S. Determining cardiac velocity fields and intraventricular pressure distribution from a sequence of ultrafast CT cardiac images. IEEE Trans Med Imaging 1994; 13(2):386-397.
Wagshul M, Eide P, Madsen J. The pulsating brain: A review of experimental and clinical studies of intracranial pulsatility. Fluids and Barriers of the CNS 2011; 8:5

### LIST OF REFERENCE SIGNS

10 Imaging apparatus
20 Computer apparatus
30 Memory
100 Program step
110 Program step
120 Program step
M(t) Measurement signal
φ(t) Signal phase
u Vector field
G(t) Motion encoding gradient
∇·u Divergence of the vector field
p Pressure

1. A method for characterizing tissue of human or animal tissue, wherein
a vector field (u) is established, which specifies the mechanical deflection or a time derivative of the mechanical deflection of tissue particles present in the tissue,
the divergence of the vector field (∇·u) is determined and
the divergence of the vector field is used as a measurement result characterizing the tissue for the purposes of tissue characterization.

2. The method as claimed in claim 1, wherein
using the divergence of the vector field as a measurement result, at least one measurement value (p) is established, which describes a local volume change in the examined tissue.

3. The method as claimed in claim 1, wherein
using the divergence of the vector field as a measurement result, at least one measurement value (p) is established, which has the dimension of pressure.

4. The method as claimed in claim 1, wherein
the phase of a measurement signal (M(t)) from an imaging apparatus (10) is evaluated and the vector field is determined using the phase signal (φ).

5. The method as claimed in claim 1, wherein the measurement value specifies a local volume or pressure change in the tissue which is based on an intrinsic pressure change.

6. The method as claimed in claim 1, wherein
the tissue is stimulated externally and
the measurement value specifies a local volume or pressure change in the tissue based on the external simulation.

7. The method as claimed in claim 1, wherein
the measurement value is formed by multiplying the magnitude of the divergence of the vector field by a proportionality factor.

8. The method as claimed in claim 1, wherein
the measurement value is formed by solving an integral equation which contains the divergence of the vector field as part of the integrand of an integral.

9. The method as claimed in claim 1, wherein the phase of a magnetic resonance imaging measurement signal is evaluated and the vector field is determined using this phase signal and/or
an ultrasound signal is generated and coupled into the tissue to be examined and the vector field is established by measuring and evaluating a measured back-coupled ultrasound signal.

10. An arrangement for characterizing tissue of human or animal tissue, characterized by
a computer apparatus and memory,
wherein a program for controlling the computer apparatus is stored in the memory and
wherein the computer apparatus—when executing the program—is suitable for determining the divergence of a vector field (u), which specifies the mechanical deflection or a time derivative of the mechanical deflection of tissue particles present in the tissue, and for using the divergence of the vector field as a measurement result characterizing the tissue for the purposes of tissue characterization.
